(19) United States
(12) Patent Application Publication
(10) Pub. No.: US 2016/0244829 A1
(43) Pub. Date: Aug. 25, 2016

(54) METHOD FOR TARGET DNA ENRICHMENT USING CRISPR SYSTEM

(71) Applicant: UNIVERSITY-INDUSTRY FOUNDATION, YONSEI UNIVERSITY, Seoul (KR)

(72) Inventors: Duhee BANG, Seoul (KR); Ji Won LEE, Seoul (KR); Hyeon Seob LIM, Chungcheongnam-do (KR)

(21) Appl. No.: 15/053,859

(22) Filed: Feb. 25, 2016

(30) Foreign Application Priority Data
Feb. 25, 2015 (KR) 10-2015-0026203

(51) Int. Cl. C12Q 1/68

(52) U.S. Cl. CPC C12Q 1/6874; C12Q 1/6806

(57) ABSTRACT

The present invention relates to a method of capturing a target nucleic acid sequence in genome sequencing, e using a CRISPR system. According to the present invention, the use of a plurality of CRIPSR systems enables capturing a plurality of target nucleic acids within genome simultaneously.

### CROSS-REFERENCE TO RELATED APPLICATION

This application claims priority to and the benefit of Korean Patent Application No. 10-2015-0026203, filed on Feb. 25, 2015, the disclosure of which is incorporated herein by reference in its entirety.

### SEQUENCE STATEMENT

Incorporated by reference herein in its entirety is the Sequence Listing entitled “G16U16C0004P.US_seq_prj_ST25,” created Feb. 25, 2016, size of 30 kilobyte.

### TECHNICAL FIELD

The technique disclosed in the present specification generally relates to a method of capturing a target nucleic acid sequence in genome sequencing.

### BACKGROUND ART

Generally, the capturing of a nucleic acid sequence used in genome sequencing is performed by the following methods. First is a selective amplification method using an oligonucleotide which is a single-stranded DNA, second is a genetic sequence cutting method using a restriction enzyme, third is a selective amplification method using a molecular inversion probe (MIP), and the last is a capturing method using RNA hybridization.

Among them, the selective amplification method using an oligonucleotide is a method in which an oligonucleotide which is referred to as a primer that has the same sequence as both ends of a sequence to be amplified is prepared and undergoes a polymerization reaction with a DNA polymerase and dNTPs (dATP, dTTP, dCTP, dGTP) for a selective amplification of only the region to be captured in the middle of the genetic sequence. This method may be easy to use when there are only a few regions to be captured, but when there are a large number of regions to be captured, numerous oligonucleotides are required. In this case, there is a disadvantage in that the individual oligonucleotides mutually interfere such that they all are not amplified satisfactorily. In addition, primer sequences differ depending on the regions to be amplified, resulting in different binding affinities between a DNA and the primer during a polymerization reaction. Therefore, the amplification efficiency differs by the regions to be amplified, and it is impossible to achieve uniform amplifications.

Next, the method of capturing a target genetic sequence using a restriction enzyme makes use of a characteristic of the restriction enzyme to cut at a particular site by recognizing only a particular genetic sequence. Therefore, it is possible to cut out only the region to be captured, as long as the sequence recognizable by the restriction enzyme exists in the region to be captured. However, the method has a disadvantage in that it cannot be used when the sequence recognizable by the restriction enzyme does not exist in the vicinity of the sequence to be captured. Also, when two or more restriction enzymes are used, a common working buffer suitable for those restriction enzymes needs to be selected because enzyme activities differ depending on the buffer. Therefore, like the selective amplification method using oligonucleotides, with increasing number of regions to be captured, it becomes increasingly difficult to use this method.

The relatively recently developed method of selective amplification using MIP is a method in which long oligonucleotides with an inverted central region are bound to both ends of a genetic sequence to be captured and the region between both ends is amplified. The method which overcomes the disadvantages of other methods to a large extent enables capturing of a genetic sequence nearly without mutual interference even when thousands or tens of thousands of types of oligonucleotides are used during the capturing process. However, in this method, the binding affinity to a DNA differs again by the binding sequence of MIP, causing differences in binding efficiency of MIP depending on the regions to be captured. Accordingly, differences in efficiency occur depending on the regions to be captured such that capturing is not uniformly achieved.

Lastly, the RNA hybridization method is a method in which an RNA (to which biotin is bound in advance) is bound to a DNA to be captured and then is separated again from the DNA using the biotin, based on the fact that a binding affinity between DNA-RNA is stronger than a binding affinity between DNA-DNA. It is a method with the highest efficiency among the methods developed thus far, but there are disadvantages in that the capturing process is complicated and that capturing efficiency decreases with the regions to be captured becoming smaller.

Meanwhile, CRISPR system is an immune system of a prokaryotes or archaeas. Recently, lots of studies regarding use of CRISPR system for gene editing, increased rapidly (Jinek et al, A Programmable Dual-RNA-Guided DNA Endonuclease in Adaptive Bacterial Immunity, Science, 2012), (Zalatan et al, Engineering Complex Synthetic Transcriptional Programs with CRISPR RNA Scaffolds, Cell, 2014). However, there is no report regarding use of CRISPR system for use in capturing a target nucleic acid sequence in genome sequencing.

### DISCLOSURE

### Technical Problem

Therefore, the present invention is directed to providing a new method of simultaneously and efficiently capturing a plurality of target nucleic acid sequence in genome sequencing.

### Technical Solution

Hence, the present invention provides a method of simultaneously capturing a plurality of target nucleic acid sequence which are located at multiple sites in genome, using a CRISPR (clustered regularly-interspaced short palindromic repeats) system.

The CRISPR system is mostly an immune system of a prokaryotes or archaeas, which provides resistance to foreign invaders such as viruses, and usually classified in four types, type I, type II, type III, and type U.

To use a type II CRISPR system which is best known among the above as an example, a CRISPR-Cas complex which is a combination of a Cas protein bound to an RNA complex consisting of a CRISPR RNA (crRNA) and a trans-activating crRNA (tracrRNA) recognizes and cuts out a specific location of target sequence. The CRISPR-Cas complex is known to recognize a target sequence which is approximately the first 20 bps (base pairs) of a specific sequence referred to as PAM and to cut at a specific site within or nearby the target sequence. In addition, since a sgRNA (single guide RNA) which is a chimeric form of crRNA and tracrRNA also discovered to play the same role as the complex of crRNA and tracrRNA, it is also well known that a complex of sgRNA and a CRISPR enzyme can cut out a target sequence.

Introducing specific mutations at DNA cleavage domains of Cas proteins causes functional loss of DNA cleavage. For example, introducing both D10A and H840A mutations to Cas9 protein from Streptococcus pyogenes causes functional loss of double strand DNA cleavage and called dead Cas9(dCas9). Also, introducing D10A or H840A mutation to Cas9 protein causes functional loss of each single strand DNA cleavage.

The inventors paid attention to the fact that if we just undergo a designing process of the sgRNA for the target sequence, we can use the CRIPSR system to cut out or attach to the specific sequence relatively freely and noted that if we use a plurality of CRIPSR systems, a plurality of desired sequence regions for genome sequencing can be captured simultaneously by simply cutting out a desired sequence region or complimentarily binding to a desired sequence region. Hence, the present invention provides

A method of capturing a target nucleic acid sequence in genome sequencing, the method comprising:

treating a genome sample including a target nucleic acid sequence, with a plurality of CRISPR systems that can cut at both ends of the target nucleic acid sequence or can complementarily bind to CRISPR complex-binding sequence within the target nucleic acid sequence, and

sorting the target nucleic acid sequence from fragments of genome sample or PCR amplification products thereof,

wherein one or more target nucleic acid sequences within genome are captured simultaneously.

“CRISPR systems” slightly differ in composition by types (type I, type II, type III, and type U) but include CRISPR enzyme and RNA that binds to the CRISPR enzyme in common.

In the present specification, the “CRISPR system” refers to a combination of CRISPR enzyme including wild type CRISPR enzyme and mutated CRISPR enzyme, CRISPR system RNAs including crRNA:tracrRNA complex or sgRNA or derivatives thereof and other additional elements required for the operation of CRISPR system.

In the present specification, the “CRISPR enzyme” is also referred as “CRISPR Associated (Cas) enzyme”. In the same line, the “CRISPR system” is used interchangeably with “a CRISPR complex” or “a CRISPR-Cas complex”.

Inside CRISPR system, CRISPR enzyme forms a complex with CRISPR system RNAs and the complex hybridize to CRISPR system binding sequence within a target nucleic acid sequence.

The CRISPR enzyme is sometimes also referred by a name other than Cas enzyme depending on the microorganism from which the CRISPR system originates. Functionally different CRISPR enzymes such as nickase CRISPR enzyme with one mutation among cleavage domains, and non-cleavable CRISPR enzyme, also called dead CRISPR enzyme with two or more mutations at each cleavage domains are well known. In the present invention, the “CRISPR enzyme” includes “wild type CRISPR enzyme” and “mutated CRISPR enzyme”. “Wild type CRISPR enzyme” refer to an enzyme that can bind to CRISPR complex-binding sequence and cut a predetermined sequence within CRISPR complex-binding sequence or around thereof. On the other hand, “mutated CRISPR enzyme” means an enzyme that can bind to CRISPR complex-binding sequence, but lost its cutting ability in whole or in part. In the following examples, “Cas9 enzyme” was used as a wild type CRISPR enzyme, and “dCas9 enzyme” was used as a “mutated CRISPR enzyme”, respectively.

Also, “CRISPR system RNAs” includes crRNA:tracrRNA complex, sgRNA, or derivatives thereof.

The CRISPR systems mutually differ in terms of the type of the CRISPR enzyme and although in same CRISPR system type, amino acid sequences of CRIPR enzymes are different depending on the species of a microorganism from which the systems originate. Also, the sequence of a crRNA, a tracrRNA, and a chimeric sgRNA are varying depending on the systems originate.

Those skilled in the art may select and use what is suitable among CRISPR systems from various microorganisms in consideration of the capturing efficiency, accuracy, and the like.

In addition, even when the CRISPR system is not from a single microorganism species, it is also possible to use a combination of CRISPR enzymes with CRISPR system RNAs that originates from various microorganisms, as long as it enables the operation of the CRISPR system that makes efficient and accurate capturing possible.

The present invention is characterized by the simultaneous capture of target nucleic acid sequences located at multiple sites within genome, by utilizing a plurality of CRISPR systems or the CRISPR complex for two or more target nucleic acid sequences.

In the present invention, the CRISPR system that is used for capturing target nucleic acid sequences may employ CRISPR enzymes along with a plurality of sets of a CRISPR system RNAs.

In the present invention, “target nucleic acid sequence” is used as a term that is distinguished from “CRISPR complex-binding sequence”. While the “CRISPR complex-binding sequence” refers to a specific sequence that a CRISPR system recognizes and cuts or attaches, the “target nucleic acid sequence” refers to a nucleic acid sequence that is obtained as a result of cutting the specific sequence of the “CRISPR complex-binding sequence” or attaching to the specific site of the “CRISPR complex-binding sequence” by utilizing a plurality of CRISPR complexes

The method of capturing a target nucleic acid sequence according to the present invention includes the following two methods: 1) a capturing method based on cutting nucleic acid sequences, and 2) a capturing method based on complementary binding to CRISPR complex-binding sequences.

With respect to the first method, an embodiment of the present invention provides a method of capturing a target nucleic acid sequence in genome sequencing, the method comprising:

treating a genome sample including a target nucleic acid sequence, with a plurality of CRISPR systems that can cut at both ends of the target nucleic acid sequence,

sorting the target nucleic acid sequence from fragments of genome sample or PCR amplification products thereof,

wherein one or more target nucleic acid sequences within genome are captured simultaneously.

To aid the understanding of the above embodiment, the schematic view of FIG. 1 can be used as an example. FIG. 1 schematically illustrates CRISPR complexes simultaneously cutting at multiple sites within a specific target sequences and sort target nucleic acid sequences. To sort target nucleic acid sequences from nucleic acids, CRISPR complexes are formed after mixing CRISPR enzyme and CRISPR system RNA library and the complexes recognize and cleave multiple target sequences depends on each CRISPR system RNA.

FIG. 2 is a schematically illustrates two CRISPR complexes (I, II) cutting at two sites within a specific target sequences. The regions to which CRISPR system RNAs are complementarily bound are “CRISPR complex-binding sequences”, and the parts marked as a and b that are cut by “lightning bolts” represent the positions of specific sequences that are cut within the CRISPR complex-binding sequences. The “target nucleic acid sequence” that is mentioned in the present invention refers to a region between the positions within the CRISPR complex-binding sequence that are cut, that is, to a region between a and b in FIG. 2. In another embodiment of the present invention, the present invention provides a method of capturing a target nucleic acid sequence in genome sequencing, the method comprising:

treating a genome sample including a target nucleic acid sequence, with a plurality of CRISPR systems that can cut at both ends of the target nucleic acid sequence,

sorting the target nucleic acid sequence from fragments of genome sample or PCR amplification products thereof,

wherein one or more target nucleic acid sequences within genome are captured simultaneously.

With respect to the above embodiment, FIG. 3 can be used as an example.

the method of capturing of a nucleic acid sequence according to the present invention may be usefully employed in analyzing a genome sequence, for example, to find out the genetic sequence that an unknown nucleic acid sample contains. In this case, the nucleic acid sequence is cut into a size suitable for analyzing with a sequencing device, for example, in a range of about 300 to 500 bps, for a sequence analysis.

When the sequence to be captured is not suitable to be immediately put in the sequencing device—for example, when the sequence to be captured is too long—the capturing of the sequence to be captured may be achieved by using three or more CRISPR-Cas complexes as shown in FIG. 3. In this case, each of the three CRISPR-Cas complexes (III, IV, V) performs cutting at p, q, r, respectively, resulting in the acquisition of the target nucleic acid which corresponds to p-r. The present invention also provides a capturing method based on complementary binding to CRISPR complex-binding sequences. Regarding the above method, an embodiment of the present invention provides a method of capturing a target nucleic acid sequence in genome sequencing, the method comprising:

treating a genome sample including a target nucleic acid sequence, with a plurality of CRISPR systems that can complementarily bind to CRISPR complex-binding sequence within the target nucleic acid sequence, and

sorting the target nucleic acid sequence from fragments of genome sample or PCR amplification products thereof,

wherein one or more target nucleic acid sequences within genome are captured simultaneously.

To aid the understanding of the above embodiment, the schematic view of FIGS. 4 and 5 can be explained in detail. FIG. 4 schematically illustrates CRISPR complexes simultaneously attach to multiple specific sites within target nucleic acid sequences, and the target nucleic acid sequences that complimentarily bound to CRISPR complex are selected from the genome fragments, thereby capturing a target nucleic acid sequences. Also, FIG. 5 schematically illustrates two CRISPR complexes (VI, VII) attaching at two sites (marked VI and VII) in a specific sequence of a polynucleotide to capture a target nucleic acid sequences VI and VII.

In case of FIGS. 4 and 5, CRISPR complex with the mutated CRISPR enzyme can form complementary binding with CRISPR-binding sequence, however, the mutated CRISPR enzyme cannot cleavage a specific site within CRISPR-binding sequence. CRISPR complexes that bound to target nucleic acid sequences through CRISPR-binding sequence can be sorted by using well-known techniques, thereby finally isolating target nucleic acid sequences. In the above, target nucleic acid sequences means the sorted nucleic acid sequences in below of FIG. 4.

Nucleic acids containing target nucleic acid sequences can be randomly fragmented by know shearing methods such as sonication or transposon tagmentation before or after CRISPR complex attachment but not limited thereto. For example, sonification may be used in case that shearing is performed before CRISPR complex attachment and transposon tagmentation may be used in case that shearing is performed after CRISPR complex attachment, but not limited thereto. FIG. 4 schematically illustrates genome sample is randomly fragmented before treating genome sample with CRISPR complex.

Meanwhile, a Cas9 enzyme is a representative CRISPR enzyme. The Cas9 enzymes differ slightly depending on the species of microorganism from which it originates. In the present invention, the Cas9 enzyme includes an ortholog of Cas9 and mutant form of Cas9. An example of such a Cas9 enzyme may be an ortholog of Cas9 derived from the genus of a microorganism selected from the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter but not limited thereto.

In the present invention, the CRISPR enzyme may be a wild type, or it may contain one or more mutations. Mutated CRISPR enzymes include nickase CRISPR enzyme which cut off one strand from double strand DNA and dead CRISPR enzyme which can attach to target sequence but loss of cut off ability.

According to one specific exemplary embodiment, the CRISPR enzyme used in the present invention may be a Cas9 enzyme.

Such a CRISPR enzyme, may be synthesized by a common protein synthesis method known to those skilled in the art and purified for use. For example, the CRISPR enzyme may be prepared by protein preparation methods including overexpression in E. coli, solid-phase synthesis, etc.

In addition, it is necessary to use a “working buffer” which causes the CRISPR system to show activity in order that the CRISPR enzyme works during the capturing of the target nucleic acid sequence according to the present invention. Conditions of a working buffer for a CRISPR system are well known in the art.

In the meantime, the CRISPR system RNAs, which form(s) a CRISPR complex by combining with a CRISPR enzyme, may be determined by the type of the CRISPR enzyme. CRISPR complex-binding sequences means a region that the CRISPR complex binds a sequence of about the first 10 bps or more of a specific sequence that exist in the upstream of so-called PAM sequence. The PAM sequence varies in sequence and length depending on the species of microorganism from which the CRISPR complex originates, and the detailed sequence thereof is well known in the art (Shah, et al, Protospacer recognition motifs: mixed identities and functional diversity, RNA biology, 2013). When selecting a suitable one among the CRISPR systems from various microorganisms, the PAM sequence is also determined Sequences of CRISPR system RNAs which can cut off or attach to target sequence and recognize the PAM sequence is also determined depending on the microorganism from which the CRISPR system originates. Determined CRISPR system RNAs can be used for CRISPR system.

Meanwhile, the tracrRNA serves to connect the crRNA and the CRISPR enzyme. The sequence information of tracrRNA, crRNA and derivatives thereof are also known for various origins of the CRISPR complexes.

In addition, among the CRISPR system RNAs, the sgRNA which is chimeric form of crRNA:tracrRNA combined into one sequence includes target sequence-binding region (corresponding to CRISPR complex-binding sequence) and scaffold region. Since the information on the scaffold region for various origins of the CRISPR complexes is partly disclosed, those skilled in the art may be able to synthesize a CRISPR system RNAs by choosing appropriate sequence information.

The method of simultaneously capturing genetic sequences which are located at multiple sites according to the present invention may be able to simultaneously capture one, several, dozens, hundreds, thousands, tens of thousands, hundreds of thousands, or millions of sequences to be captured. For this, a sgRNA pool containing individual sgRNAs for various sequences to be captured may be used in the present invention.

In one specific exemplary embodiment, the CRISPR system RNA, sgRNA in this case, may be obtained from a template DNA by in vitro transcription but is not thereby limited. The template DNA which is used for the acquisition of the sgRNA includes: a promoter that can bind with RNA polymerase to initiate transcription, a DNA sequence (i.e. a target sequence) that codes the sgRNA, and a sgRNA scaffold. Since the promoter and the sgRNA scaffold are common for all sgRNAs contained in the sgRNA pool, it is sufficient that the template DNA is synthesized by varying only the target sequence.

For example, the template DNA may be prepared by a microarray oligonucleotide synthesis method but is not thereby limited. Specifically, the exemplary preparation by a microarray oligonucleotide synthesis method may be carried out by fixing a library of the template DNA that corresponds to a library of the desired CRISPR system RNAs, in this case sgRNA, on a microchip for a synthesis and subsequent cutting. The sgRNA library is obtained by in vitro transcription from the template DNA synthesized as in the above.

The schematic view of FIG. 1 illustrates a process by which target nucleic acids are captured by CRISPR-Cas complexes that are formed by configuring various sgRNA libraries and subsequently hybridize to target sequence and cut off target nucleic acid sequences.

The schematic view of FIG. 2 illustrates a process by which target nucleic acids are captured by CRISPR-Cas complexes that are formed by configuring various sgRNA libraries and subsequently hybridize to target sequence and attach to target nucleic acid sequences.

In capturing a specific nucleic acid sequence by applying the present invention, the type or origin of the target nucleic acid sequence is not particularly limited. In another specific exemplary embodiment, the target nucleic acid sequence may originate from an animal or a plant. Also target nucleic acid sequence may be any of DNA, RNA, or PNA.

In another specific exemplary embodiment, the target nucleic acid sequence may originate from an animal or a plant.

As explained above, in case of using cut off method, the CRISPR enzyme may be a wild type of CRISPR enzyme. On other hand, in case using only complementary binding of CRISPR system except cut off ability, the CRISPR enzyme may be a mutated CRISPR enzyme.

Further, the capture method of present invention comprises a step for sorting target nucleic acid sequences from fragments of genome sample or PCR amplification products thereof.

The pool containing target nucleic acid sequences may be genome sample fragments or PCR amplification product. For enrichment of target nucleic acid sequences, genome sample fragments are preferable amplified by PCR, but not limited thereto.

Sorting of target nucleic acid sequence, may performed by isolating based on nucleic acid size or isolating using probe, but not limited thereto. As isolation based on nucleic acid size, a known method such as agarose gel electrophoresis may be used. Such sorted target nucleic acid sequences are conjugated with adapter sequence through known methods such as PCR or ligase, then undergo sequencing thereby confirming whether the capturing is exactly performed.

In order to sort target nucleic acid sequences using probe, probe-containing CRISPR system RNAs or probe-containing CRISPR enzymes are constructed and then CRISPR complex are purified by using those probe. For example, but not limited thereto, after cleavage or attachment of CRISPR complex to target nucleic acid sequence, many CRISPR complexes stay stable on those target sequences. Constructing CRISPR complex with biotinylated CRISPR system RNAs, enables purifying CRISPR complex with magnetic streptavidin-biotin binding. The other way is construct CRISPR complex with CRISPR enzyme containing 6× histidine tag. After CRISPR complex cleave or attach to target nucleic acid sequence, those stable hybridized complexes can be purified with 6× histidine tag using Ni-NTA. For sorting target nucleic acid, type of probe and bead biding with probe are well known in the art.

In case of sorting target nucleic acid sequences using probe, there may be additional step for dissociation of a target nucleic acid sequence from CRISPR complex. There are well known methods for dissociation of a nucleic acid from enzyme. For example, a target nucleic acid sequence can be dissociated from CRISPR complex by adding 0.2% Sodium Dodecyl Sulfate(SDS) solution to a solution comprising a target nucleic acid sequence bound to CRISPR complex since the CRISPR enzyme lost its enzymatic function due to SDS, but not limited thereto.

Hereinafter, the present invention will be described in detail through examples. The following examples are merely provided to illustrate the present invention, and the scope of the present invention is not limited to the following examples. The examples are provided to complete the disclosure of the present invention and to fully disclose the scope of the present invention to those of ordinary skill in the art, and the present invention is only defined by the range of the appended claims.

### Advantageous Effects

According to the present invention, the use of a plurality of CRISPR systems enables capturing a plurality of target nucleic acids within genome simultaneously.

### DESCRIPTION OF DRAWINGS

FIG. 1 is a schematic view showing a process by which target nucleic acid sequences are captured (cleaved) by CRISPR system RNA library and CRISPR enzyme complex from whole nucleic acids containing target nucleic acid sequences and sorting target nucleic acid sequences.

FIG. 2 is a schematic view showing two CRISPR complexes (I, II) cutting at two sites (marked a and b) in a specific sequence of a polynucleotide to capture a target nucleic acid sequence (the sequence between a and b).

FIG. 3 is a schematic view showing three CRISPR complexes (III, IV, V) cutting at three sites (marked as p, q, and r) in a specific sequence of a polynucleotide to capture target nucleic acid sequences (the sequences between p and r).

FIG. 4 is a schematic view showing a process by which target nucleic acid sequences are captured (attached) by CRISPR system RNA library and CRISPR enzyme complex from whole nucleic acids containing target nucleic acid sequences that are sheared before or after attachment.

FIG. 5 is a schematic view showing two CRISPR complexes (VI, VII) attaching at two sites (marked VI and VII) in a specific sequence of a polynucleotide to capture a target nucleic acid sequences VI and VII.

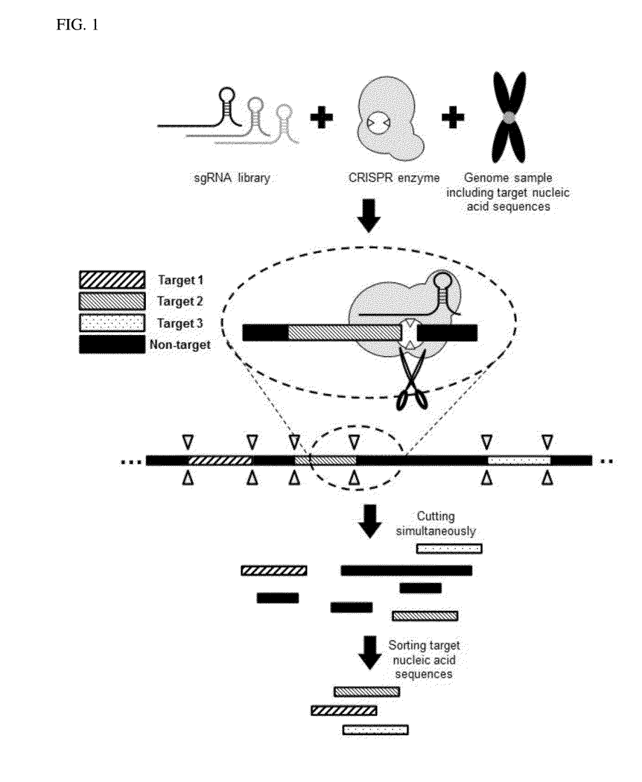

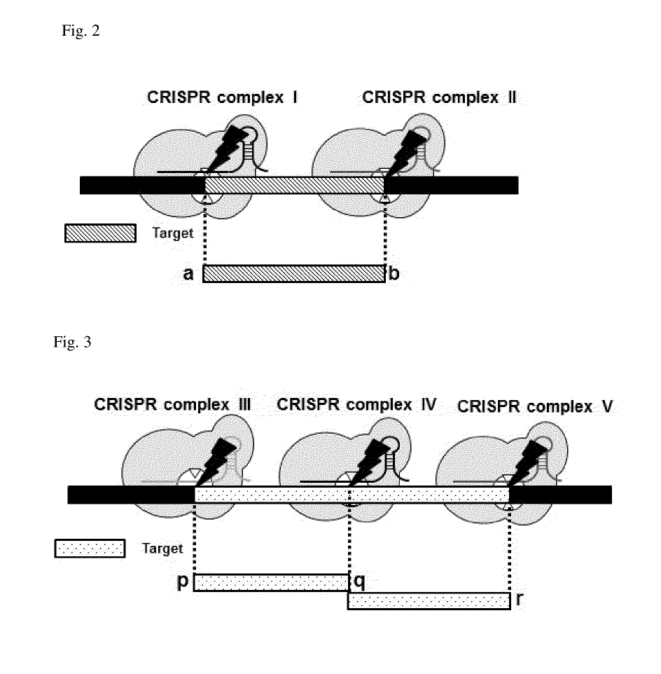

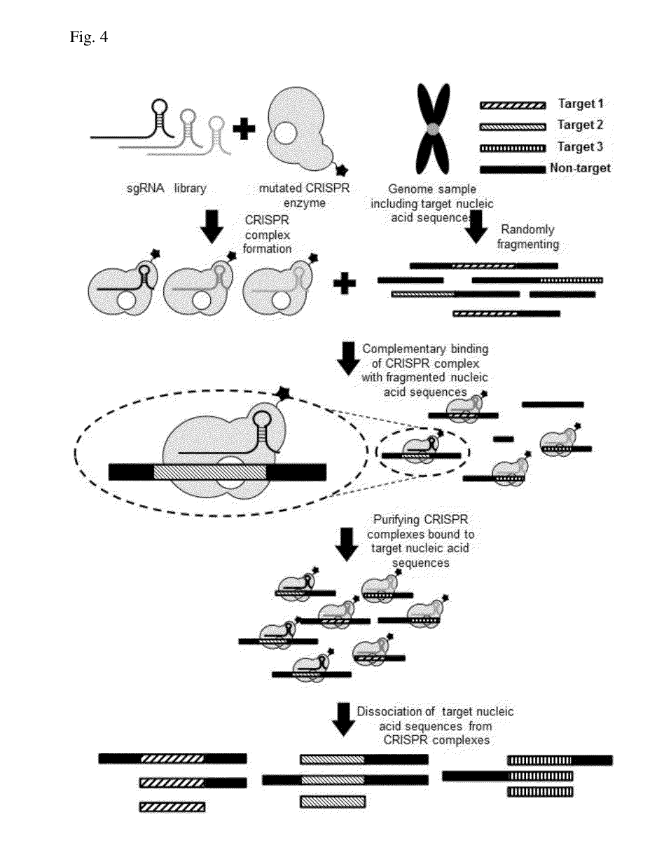

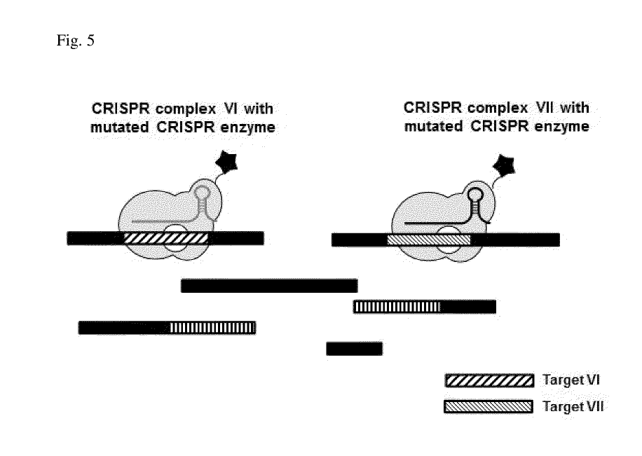

### MODES OF THE INVENTION

### Examples

### I. Capturing of a Plurality of Target Nucleic Acid Sequences Based on Cleavage of CRISPR System

### Preparation Example 1

### Design and Preparation of CRISPR System RNAs for Capturing Genetic Sequences Located at Multiple Sites by Cleaving DNAs

CRISPR system RNAs used in the present invention are sgRNA. sgRNAs for cleaving both ends of target nucleic acid sequences are designed to recognize the upstream 18 bps of the base PAM sequence of a target region. In the present exemplary embodiment, ‘NGG’ (N=one of A, T, C, and G) was used as the PAM sequence. The NGG sequence is a PAM sequence that streptococcus pyogenes specifically recognizes, and it is sufficient that a random base among A, T, C, G is positioned ahead of GG.

The sgRNA whose binding site is designed as in the above was obtained from a template DNA by an in vitro transcription, and for this, the template DNA was combined with an sgRNA template sequence and a T7 promoter with 6 bp gap sequence which can initiate a transcription by binding with a T7 RNA polymerase. In this case, the T7 promoter employed has a sequence of ‘GGATTCTAATACGACTCACTATAGG’ (SEQ ID NO: 1), and an sgRNA scaffold which is the sgRNA template sequence other than an 18-bp sequence that binds with the target nucleic acid has the following sequence:

(SEQ ID NO: 3)
′GTTTTAGAGCTAGAAATAGCAAGTTAAAATAAGGCTAGTCCGTTATCAA
CTTGAAAAAGTGGCACCGAGTCGGTGCTTTTTTT′

An 18-bp target sequence that corresponds to ‘NNNNNNNNNNNNNNNNNN’ (N=one of A, T, C, and G) (SEQ ID NO: 2) is located between the T7 promoter sequence of the SEQ ID NO: 1 and the sgRNA scaffold of the SEQ ID NO: 3. The target sequence differs depending on the position of the genetic sequence to be cut at.

As a result, the sequence of the synthesized template DNA is the same as SEQ ID NO: 4 in which the T7 promoter, target sequence, and sgRNA template sequence are combined sequentially.

(SEQ ID NO: 4)
′GGATTCTAATACGACTCACTATAGGNNNNNNNNNNNNNNNNNNG
TTTTAGAGCTAGAAATAGCAAGTTAAAATAAGGCTAGTCCGTTATCAACT
TGAAAAAGTGGCACCGAGTCGGTGCTTTTTTT′

To prepare sgRNA that targets each of the desired regions, an in vitro transcription was carried out using a template DNA library. The transcribed sgRNA was precipitated with LiCl and prepared into pellets by centrifugation (13000 rpm, 5 min, 4° C.). The pellets were washed with 70% ethanol and subsequently precipitated again by centrifugation (13000 rpm, 5 min, 4° C.). Then, the sgRNA was dried to be completely rid of ethanol and subsequently dissolved in water (without a nuclease) for storage. The sgRNA was used at a concentration of 500 nmol to confirm a capturing ability, and 3 μg of the sgRNA library was used when capturing multiple sequences simultaneously. Immediately before capturing, the temperature of the solution containing a sgRNA was raised to 95° C. and then reduced to 37° C. at a rate of 0.1° C. per second for re-folding and use.

Some of the sgRNA contained in the sgRNA pool synthesized by the above-described process are provided as examples following:

### Preparation Example I-1-1

### Synthesis of Two sgRNAs to Capture Portion of 1448014-1448256 of Chromosome 1

To capture the portion of 1448014-1448256 (SEQ ID NO: 5) in chromosome 1, ‘GAAAGAGTCCGATCCTCCGTTTTAGAGCTAGAAATAGCAAGTTAAAATA AGGCTAGTCCGTTATCAACTTGAAAAAGTGGCACCGAGTCGGTGCTTTTT TT’ (SEQ ID NO: 7) which is an sgRNA that recognizes ‘GGAGGATCGGACTCTTTC’ (SEQ ID NO: 6) that is a portion corresponding to 1448011-1448028 was synthesized to constitute the front portion, and ‘TACGCTTCCCTTGTTACGGTTTTAGAGCTAGAAATAGCAAGTTAAAATAA GGCTAGTCCGTTATCAACTTGAAAAAGTGGCACCGAGTCGGTGCTTTTTT T’ (SEQ ID NO: 9) which is an sgRNA that recognizes ‘CGTAACAAGGGAAGCGTA’ (SEQ ID NO: 8) that is a portion corresponding to 1448254-1448271 was synthesized to constitute the end portion.

### Preparation Example I-1-2

### Synthesis of Two sgRNAs to Capture Portion of 55537908-55538174 of Chromosome 1

To capture the portion of 55537908-55538174 (SEQ ID NO: 10) in chromosome 1, ‘TCATACCTCTCTTCTCAGGTTTTAGAGCTAGAAATAGCAAGTTAAAATAA GGCTAGTCCGTTATCAACTTGAAAAAGTGGCACCGAGTCGGTGCTTTTTT T’ (SEQ ID NO: 12) which is an sgRNA that recognizes ‘TCATACCTCTCTTCTCAG’ (SEQ ID NO: 11) that is the portion corresponding to 55537893-55537910 was synthesized to constitute the front portion, and ‘TTAAAAGCATCCCAAGTAGTTTTAGAGCTAGAAATAGCAAGTTAAAATA AGGCTAGTCCGTTATCAACTTGAAAAAGTGGCACCGAGTCGGTGCTTTTT TT’ (SEQ ID NO: 14) which is an sgRNA that recognizes ‘TTAAAAGCATCCCAAGTA’ (SEQ ID NO: 13) that is a portion corresponding to 55538160-55538177 was synthesized to constitute the end portion.

### Preparation Example I-1-3

### Synthesis of Three sgRNAs to Capture Portion of 38406959-38407462 of Chromosome 10

To capture the portions of 38406959-38407462 (SEQ ID NO: 15) of chromosome 10, ‘TCAGAGAACACACACAGGGTTTTAGAGCTAGAAATAGCAAGTTAAAATA AGGCTAGTCCGTTATCAACTTGAAAAAGTGGCACCGAGTCGGTGCTTTTT TT’ (SEQ ID NO: 17) which is an sgRNA that recognizes ‘TCAGAGAACACACACAGG’ (SEQ ID NO: 16) that is a portion corresponding to 38406946-38406963 was synthesized, ‘GCATCAGAAAACACACACGTTTTAGAGCTAGAAATAGCAAGTTAAAATA AGGCTAGTCCGTTATCAACTTGAAAAAGTGGCACCGAGTCGGTGCTTTTT TT’ (SEQ ID NO: 19) which is an sgRNA that recognizes ‘GCATCAGAAAACACACAC’ (SEQ ID NO: 18) that is a portion corresponding to 38407195-38407212 was synthesized to constitute the middle portion, and ‘ACATCTGAGAAGACACACGTTTTAGAGCTAGAAATAGCAAGTTAAAATA AGGCTAGTCCGTTATCAACTTGAAAAAGTGGCACCGAGTCGGTGCTTTTT TT’ (SEQ ID NO: 21) which is an sgRNA that recognizes ‘ACATCTGAGAAGACACAC’ (SEQ ID NO: 20) that is a portion corresponding to 38407447-38407464 was synthesized to constitute the end portion.

### Preparation Example I-1-4

### Synthesis of Two sgRNAs to Capture Portion of 9580101-9580360 of Chromosome 12

To capture the portion of 9580101-9580360 (SEQ ID NO: 22) of chromosome 12, ‘ACAGGCGTGTTGCGTTAAGTTTTAGAGCTAGAAATAGCAAGTTAAAATA AGGCTAGTCCGTTATCAACTTGAAAAAGTGGCACCGAGTCGGTGCTTTTT TT’ (SEQ ID NO: 24) which is an sgRNA that recognizes ‘ACAGGCGTGTTGCGTTAA’ (SEQ ID NO: 23) that is a portion corresponding to 9580087-9580104 was synthesized to constitute the front portion, and ‘ACTTCCGAGCTTAACCCTGTTTTAGAGCTAGAAATAGCAAGTTAAAATAA GGCTAGTCCGTTATCAACTTGAAAAAGTGGCACCGAGTCGGTGCTTTTTT T’ (SEQ ID NO: 26) which is an sgRNA that recognizes ‘AGGGTTAAGCTCGGAAGT’ (SEQ ID NO: 25) that is a portion corresponding to 9580357-9580374 was synthesized to constitute the end portion.

### Preparation Example I-2

### Preparation of Cas9 Protein to Capture Genetic Sequences Located at Multiple Sites

A Cas9 gene of Streptococcus pyogenes was inserted into a pET28a vector which is a type of an E. coli expression vector. In this case, the portion of a vector sequence that is related to protein expression consists of a T7 promoter, a Cas9 gene, and a DNA sequence that expresses a histidine-tag (His-tag) for purification. This vector is a vector whose expression is controlled by a T7 RNA polymerase and a lac operator, occurs only in the presence of a T7 RNA polymerase, and increases significantly when the vector is incubated with isopropyl beta-D-1-thiogalactopyranoside (IPTG). The vector that was prepared as thus was introduced to E. coli (T7 Express Competent E. coli from NEB Inc.) having a T7 RNA polymerase to overexpress the Cas9 protein, and the protein was subsequently purified.

In purifying the Cas9 protein, first, the E. coli that overexpressed the protein was collected by centrifugation (3900 rpm, 10 mM) and the cell culture medium was completely discarded. Then, a lysis buffer (20 mM Tris-HCl at pH 8.0, 300 mM NaCl, 1 mg/mL lysozyme, 1× phenylmethylsulfonyl fluoride (PMSF)) was added in a ratio of 1 mL lysis buffer/100 mL cell culture medium, and the E. coli was resuspended to be crushed by sonication (for total of 10 minutes; one cycle consists of crushing at 40% amplitude for 10 seconds and resting for 30 seconds). After sonication, the solution was centrifuged (13000 rpm, 10 min) to obtain only a supernatant and was subsequently passed through a Ni-NTA resin to leave only a protein having His-tag on the resin. Then, the resin was washed with 5 mL of washing buffer (20 mm Tris-HCl at pH 8.0, 300 mM NaCl, 20 mm imidazole 1×PMSF) three times to remove unwanted proteins that are bound to the resin abnormally. Subsequently, only the wanted proteins were collected by passing an elution buffer (20 mm Tris-HCl at pH 8.0, 300 mM NaCl, 250 mM imidazole, 1×PMSF) 500 μL through the resin eight times to again obtain the proteins.

To use the purified proteins for capturing a genetic sequence, first, the solution should be replaced by a working buffer (50 mM Tris-HCl at pH 8.0, 200 mM KCl, 0.1 mM EDTA, 1 mM DTT, 0.5 mM PMSF, 20% glycerol) in which the proteins function. This is a process employing a dialysis method to simultaneously remove imidazole which is contained in the elution buffer in a significant amount and transfer the proteins to a solution that can keep the proteins in a more stable state. Among the eight solutions that were separately eluted, three solutions that contain eluted proteins totaling 1.5 mL were put in a dialysis cassette and then were subjected to a dialysis for 16 hours using 1 L of working buffer. The proteins that changed the composition of the solution were quantified by the Bradford assay.

### Preparation Example I-3

### Purification of Genome Sample for Capturing Target Nucleic Acid Sequences Located at Multiple Sites

For obtain a genome sample for capturing the target nucleic acid sequences located at multiple sites, human embryonic kidney 293 cells (HEK293) were cultured and subsequently purified. Culture conditions included 37° C. and incubation in Dulbecco Modified Eagle Medium containing 10% fetal bovine serum as the culture medium in 5% CO₂. The cultured cells that grew while attached to the culture dish and were taken off using a Trypsin/EDTA solution. Subsequent centrifugation (3000 rpm, 10 min) collected only the cells. Then, only genomes were purified using a DNeasy 96 Blood & Tissue Kit from QIAGEN Inc.

### Test Example I-1

### Confirmation of Capturing Ability of Cas9 Protein

To confirm the capturing ability of the purified protein, an experiment was first carried out where a 1080 bp double-stranded DNA was amplified with a pUC19 vector and cut in the middle. A 1080 bp DNA to be cut was cut into lengths of about 630 bps and 450 bps during a cutting operation. To test the above, a Cas9 protein at an aforementioned concentration, sgRNA, and 300 ng DNA to be cut were mixed with a buffer solution (final concentration at 20 μL: 50 mM Tris-HCl, 100 mM NaCl, 10 mM MgCl₂, 1 mM DTT, pH 7.9) and water to prepare a total of 20 volumes. In addition, a solution with excessive amount of the Cas9 protein and a solution mixture with excessive amount of the sgRNA were allowed to react at 37° C. for 1, 8, 16 hours to confirm the cutting ability. The result suggests that 500 nmol is a sufficient amount of the sgRNA and that the amount of the Cas9 protein is most important for the reaction. Also, it can be noted that most of the cutting reactions occur within one hour.

### Example I-1

### Simultaneous Capturing of Genetic Sequences Located at Multiple Sites by Cleaving DNAs

1000 ng of the sgRNA library prepared by the preparation example I-1 was used with 3000 ng of the Cas9 protein prepared by the preparation example 1-2 under aforementioned conditions of a Cas9 working buffer. After the volume was set to 20 μL, they were allowed to react for 1 hour at 37° C. to simultaneously capture genetic sequences located at multiple sites.

To confirm if the simultaneous capturing of genetic sequences located at multiple sites had been successful, sequencing of the captured sequence was performed. Specifically, after the reaction, the entire reaction solution was purified using a MinElute PCR Purification kit from QIAGEN Inc. Immediately after, an adapter DNA sequence for using next-generation sequencing equipment from Illumina Inc. was attached to captured sequences using a SPARK DNA sample prep kit from Enzymatics Inc. Using a USER enzyme, the DNA fragments to which adapters are attached cut uracil that existed in an adapter DNA and amplified the captured sequences using a universal sequence primer and an index sequence available from Illumina Inc. The amplified sequences were separated by size using an agarose gel and, in this case, only those of desired sizes were selected for purification using a spin column of QIAGEN Inc. Subsequently, the sequencing information was obtained using a next-generation HiSeq 2500 sequencing system.

The obtained sequencing information was analyzed by programs such as a self-produced Python program, BWA, or the like to confirm if desired sequences had been captured, and it was confirmed that desired genetic sequences had been simultaneously captured.

To exemplify some of the above, the following two sequencing results among all sequencing results confirmed that the genetic sequence of SEQ ID NO: 5 corresponding to 1448014-1448256 of chromosome 1 had been captured by two sgRNAs, which were SEQ ID NO: 7 and SEQ ID NO: 9 of the preparation example I-1-1:

(SEQ ID NO: 27)
′GGATCGGACTCTTTCCGTCACCCGTTTGCACCTCTGCAGCTGTCAG
GAGCGGGTCAGGTGCGGAAAGCGGTGCGGAGGTGGCGCTCATAGGTTAC
AGGGGTCAGGGTCTGGGGCTGGCCGTGGTCTTCAGTTACCGCCGAGCGTG
CGGGAT′
and
(SEQ ID NO: 28)
′TACAGGGGTCAGGGTCTGGGGCTGGCCGTGGTCTTCAGTTACCGCCGAG
CGTGCGGGATCCTTCTGCGCTTGCCGCCTCCACGTGGCACAGGCCAAGGC
GTGGCCAGATGGGTAGATGGGTTTGTTGGGTGGTTGCTAGCAGTTTCCAC
GT′.

In addition, the sequencing results of SEQ ID NO: 29 and SEQ ID NO: 30 confirmed that the portions in chromosome 1 that correspond to 55537908-55538174 (SEQ ID NO: 10) had been accurately captured by two sgRNAs, which were SEQ ID NO: 12 and SEQ ID NO: 14 of the preparation example I-1-2.

(SEQ ID NO: 29)
′CAGAGGTTGCAGTTTCTGAGAAACACACTGAAAATCCTCCATAAG
TGATTTAGACCACGCAAAAACAAGAGACAACTCTCACCTGAGCTGAAAT
GGTTCGCTGAAAGGTTTTTCCAGTTGATGTTTCATTAGAGACATTACTCTG
TGGTGT′
(SEQ ID NO: 30)
′GTTGATGTTTCATTAGAGACATTACTCTGTGGTGTCCAGTAATGTT
CTGACATCTGAGATGAAAGGTCAAAAATGCCATCAGAGGTGACAAATAA
GCCCCCATGGGTTCACAGTTTCTACCATTAGATATTGAGTCTTAAAAGCA
TCCCAA′

Also an accurate capturing of the portions corresponding to 38406959-38407462 (SEQ ID NO: 15) of chromosome 10, which was to be captured by three sgRNAs such as SEQ ID NO: 17, 19 and 21, was identified based on four sequencing results of the following SEQ ID NO: 31 to SEQ ID NO: 34.

(SEQ ID NO: 31)
′AGGGGGAAAACCCTATGAATGTCATGAATGTGGGAAGACCTTCTA
TAAGAATTCAGACCTCATTAAACATCAAAGAATTCATACAGGGGAGAGA
CCTTATGGATGTCATGAATGTGGGAAATCCTTCAGTGAAAAGTCAACCCT
TACTCAA′,
(SEQ ID NO: 32)
′TGGATGTCATGAATGTGGGAAATCCTTCAGTGAAAAGTCAACCCT
TACTCAACATCAAAGAACGCACACAGGGGAGAAACCATATGAATGTCAT
GAATGTGGGAAAACCTTCTCATTTAAGTCAGTCCTTACTGTGCATCAGAA
AACACAC′,
(SEQ ID NO: 33)
′ACAGGGGAGAAGCCCTATGAATGCTATGCATGTGGGAAAGCCTTT
CTCAGAAAATCAGACCTCATTAAACATCAAAGAATACACACAGGTGAAA
AACCTTATGAATGTAATGAATGTGGGAAGTCATTCTCTGAGAAGTCAACC
CTTACTA′,
(SEQ ID NO: 34)
′ATGAATGTAATGAATGTGGGAAGTCATTCTCTGAGAAGTCAACCC
TTACTAAACATCTAAGAACTCACACAGGTGAGAAACCTTATGAATGTATT
CAGTGTGGAAAATTTTTCTGCTACTACTCCGGTTTCACAGAACATCTGAG
AAGACA′

In the case of another region to be captured, which is the portion corresponding to 9580101-9580360 (SEQ ID NO: 22) of chromosome 12, two sequencing results from the following SEQ ID NO: 35 and SEQ ID NO: 36 confirmed that the desired region had been captured. Also found a difference (G→C) between the genetic sequence of a human genome 19 reference by the base 9580202 of the chromosome 12 and HEK293T genome used in an experiment.

(SEQ ID NO: 35)
′TAAGGGTTAAGTAATTACACATCTGTTTTGCTTTTTCTTCCTTCTAT
AGTCTTAACATAGTACTCTACCCACAGGTGGTGACAGGAAGGAAATTGG
ATGTGCAATGTGGAAAGGTGGAAACCTCTACCTTGAACAGGTTGATGTTG
TCGAT′
(SEQ ID NO: 36)
′GGAAAGGTGGAAACCTCTACCTTGAACAGGTTGATGTTGTCGATC
TGGCTCTGGAAGAGAAAGTCGTTGATAGTCTTCAGCTCCATCCCTGAGAA
CAAACACATGAAGGGCCTTGGGAGCTTCACCCTAAGCCTCAGGTTTCAGT
CCCAGG′

As shown in the results, the simultaneous capturing of a variety of genetic sequences was successfully achieved.

### II. Capturing of a Plurality of Target Nucleic Acid Sequences Based on Complementary Binding of CRISPR

### Preparation Example II-1

### Design and Preparation of CRISPR System RNas for Capturing Genetic Sequences Located at Multiple Sites by Attaching to DNAs

CRISPR system RNAs used in the present invention are sgRNA. sgRNAs for attaching inside of target nucleic acid sequences are designed to recognize the upstream 20 bps of the base PAM sequence of a target region. In the present exemplary embodiment, ‘NGG’ (N=one of A, T, C, and G) was used as the PAM sequence. The NGG sequence is a PAM sequence that streptococcus pyogenes specifically recognizes, and it is sufficient that a random base among A, T, C, G is positioned ahead of GG.

The sgRNA whose binding site is designed as in the above was obtained from a template DNA by an in vitro transcription, and for this, the template DNA was combined with an sgRNA template sequence and a T7 promoter with 6 bp gap sequence which can initiate a transcription by binding with a T7 RNA polymerase. In this case, the T7 promoter employed has a sequence of ‘GGATTCTAATACGACTCACTATAGG’ (SEQ ID NO: 1), and an sgRNA scaffold which is the sgRNA template sequence other than an 18-bp sequence that binds with the target nucleic acid has the following sequence:

(SEQ ID NO: 3)
′GTTTTAGAGCTAGAAATAGCAAGTTAAAATAAGGCTAGTCCGTTATCAA
CTTGAAAAAGTGGCACCGAGTCGGTGCTTTTTTT′

An 20-bp target sequence that corresponds to NNNNNNNNNNNNNNNNNNNN′ (N=one of A, T, C, and G) (SEQ ID NO: 37) is located between the T7 promoter sequence of the SEQ ID NO: 1 and the sgRNA scaffold of the SEQ ID NO: 3. The target sequence differs depending on the position of the genetic sequence to be cut at.

As a result, the sequence of the synthesized template DNA is the same as SEQ ID NO: 38 in which the T7 promoter, target sequence, and sgRNA template sequence are combined sequentially.

(SEQ ID NO: 38)
GGATTCTAATACGACTCACTATAGGNNNNNNNNNNNNNNNNNNN
NGTTTTAGAGCTAGAAATAGCAAGTTAAAATAAGGCTAGTCCGTTATCAA
CTTGAAAAAGTGGCACCGAGTCGGTGCTTTTTTT′

To prepare sgRNA that targets each of the desired regions, an in vitro transcription was carried out using a template DNA library. The transcribed sgRNA was treated TURBO DNase (Ambion, Inc.) at 37° C. 15 minutes. After removing DNA template, sgRNA were purified by Oligo Clean & Concentrator™ (Zymo research Inc) 5 min, 4° C.) and dissolved in water (without a nuclease) for storage. The sgRNA library was used at 480.7 ng when capturing multiple sequences simultaneously. Immediately before capturing, the temperature of the solution containing a sgRNA was raised to 95° C. and then reduced to 37° C. at a rate of 0.1° C. per second for re-folding and use.

Some of the sgRNA contained in the sgRNA pool synthesized by the above-described process are provided as examples following:

### Preparation Example II-1-1

### Synthesis of 11 sgRNAs to Capture Bla Gene in EcNR2 Genome

To capture the bla gene′ ATGAGTATTCAACATTTCCGTGTCGCCCTTATTCCCTTTTTTGCGGCATTT TGCCTTCCTGTTTTTGCTCACCCAGAAACGCTGGTGAAAGTAAAAGATGC TGAAGATCAGTTGGGTGCACGAGTGGGTTACATCGAACTGGATCTCAAC AGCGGTAAGATCCTTGAGAGTTTTCGCCCCGAAGAACGTTTTCCAATGAT GAGCACTTTTAAAGTTCTGCTATGTGGCGCGGTATTATCCCGTATTGACG CCGGGCAAGAGCAACTCGGTCGCCGCATACACTATTCTCAGAATGACTTG GTTGAGTACTCACCAGTCACAGAAAAGCATCTTACGGATGGCATGACAG TAAGAGAATTATGCAGTGCTGCCATAACCATGAGTGATAACACTGCGGC CAACTTACTTCTGACAACGATCGGAGGACCGAAGGAGCTAACCGCTTTTT TGCACAACATGGGGGATCATGTAACTCGCCTTGATCGTTGGGAACCGGA GCTGAATGAAGCCATACCAAACGACGAGCGTGACACCACGATGCCTGTA GCAATGGCAACAACGTTGCGCAAACTATTAACTGGCGAACTACTTACTCT AGCTTCCCGGCAACAATTAATAGACTGGATGGAGGCGGATAAAGTTGCA GGACCACTTCTGCGCTCGGCCCTTCCGGCTGGCTGGTTTATTGCTGATAA ATCTGGAGCCGGTGAGCGTGGGTCTCGCGGTATCATTGCAGCACTGGGGC CAGATGGTAAGCCCTCCCGTATCGTAGTTATCTACACGACGGGGAGTCAG GCAACTATGGATGAACGAAATAGACAGATCGCTGAGATAGGTGCCTCACTGAT TAAGCATTGGTAA′ 817737-818597 (SEQ ID NO: 39) in EcNR2 genome, inventors extend 150 base pair at both ends of bla gene ‘TTATTCGGCCTTGAATTGATCATATGCGGATTAGAAAAACAACTTAAAT GTGAAAGTGGGTCTTAACAGTTCCTGGATATCCGGATGAAGGCACGAAC CCAGTGGACATAACCCTGATAAATGCTTCAATAATATTGAAAAAGGAAG AGTATGAGTATTCAACATTTCCGTGTCGCCCTTATTCCCTTTTTTGCGGCA TTTTGCCTTCCTGTTTTTGCTCACCCAGAAACGCTGGTGAAAGTAAAAGA TGCTGAAGATCAGTTGGGTGCACGAGTGGGTTACATCGAACTGGATCTCA ACAGCGGTAAGATCCTTGAGAGTTTTCGCCCCGAAGAACGTTTTCCAATG ATGAGCACTTTTAAAGTTCTGCTATGTGGCGCGGTATTATCCCGTATTGA CGCCGGGCAAGAGCAACTCGGTCGCCGCATACACTATTCTCAGAATGACT TGGTTGAGTACTCACCAGTCACAGAAAAGCATCTTACGGATGGCATGAC AGTAAGAGAATTATGCAGTGCTGCCATAACCATGAGTGATAACACTGCG GCCAACTTACTTCTGACAACGATCGGAGGACCGAAGGAGCTAACCGCTTT TTTGCACAACATGGGGGATCATGTAACTCGCCTTGATCGTTGGGAACCGG AGCTGAATGAAGCCATACCAAACGACGAGCGTGACACCACGATGCCTGT AGCAATGGCAACAACGTTGCGCAAACTATTAACTGGCGAACTACTTACTC TAGCTTCCCGGCAACAATTAATAGACTGGATGGAGGCGGATAAAGTTGC AGGACCACTTCTGCGCTCGGCCCTTCCGGCTGGCTGGTTTATTGCTGATA AATCTGGAGCCGGTGAGCGTGGGTCTCGCGGTATCATTGCAGCACTGGG GCCAGATGGTAAGCCCTCCCGTATCGTAGTTATCTACACGACGGGGAGTC AGGCAACTATGGATGAACGAAATAGACAGATCGCTGAGATAGGTGCCTC ACTGATTAAGCATTGGTAATTTGTCCACTACGTGAAAGGCGAGATCACCA AGGTAGTCGGCAAATAATGTCTAACAATTCGTTCAAGCCGACGGATATCG AGCTCGCTTGGACTCCTGTTGATAGATCCAGTAATGACCTCAGAACTCCA TCTGGATTTGTTCAGAACG’ 817587-818747(SEQ ID NO: 40) in EcNR2EcNR2 genome for sufficiently capture both ends of gene and design 11 sgRNAs in the extended bla region for binding CRISPR-Cas complex. ‘AAACAACTTAAATGTGAAAGGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 42) which is an sgRNA that recognizes ‘AAACAACTTAAATGTGAAAG’(SEQ ID NO: 41) that is a portion corresponding to 817623-817642 was synthesized to constitute the front portion, and ‘TGCTTCAATAATATTGAAAAGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 44) which is an sgRNA that recognizes ‘TGCTTCAATAATATTGAAAA’ (SEQ ID NO: 43) that is a portion corresponding to 817708-817727 was synthesized, and ‘TTTTGCTCACCCAGAAACGCGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 46) which is an sgRNA that recognizes ‘TTTTGCTCACCCAGAAACGC’(SEQ ID NO: 45) that is a portion corresponding to 817799-817818 was synthesized, and ‘CGAAGAACGTTTTCCAATGAGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 48) which is an sgRNA that recognizes ‘CGAAGAACGTTTTCCAATGA’ (SEQ ID NO: 47) that is a portion corresponding to 817916-817935 was synthesized, and ‘CATACACTATTCTCAGAATGGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 50) which is an sgRNA that recognizes ‘CATACACTATTCTCAGAATG’ (SEQ ID NO: 49) that is a portion corresponding to 818012-818031 was synthesized, and ‘TAACCATGAGTGATAACACTGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 52) which is an sgRNA that recognizes ‘TAACCATGAGTGATAACACT’ (SEQ ID NO: 51) that is a portion corresponding to 818110-818129 was synthesized, and ‘TGATCGTTGGGAACCGGAGCGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 54) which is an sgRNA that recognizes ‘TGATCGTTGGGAACCGGAGC’ (SEQ ID NO: 52) that is a portion corresponding to 818216-818235 was synthesize, and ‘ACGTTGCGCAAACTATTAACGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 56) which is an sgRNA that recognizes ‘ACGTTGCGCAAACTATTAAC’ (SEQ ID NO: 55) that is a portion corresponding to 818295-818314 was synthesize, and ‘GCTGGCTGGTTTATTGCTGAGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 58) which is an sgRNA that recognizes ‘GCTGGCTGGTTTATTGCTGA’ (SEQ ID NO: 57) that is a portion corresponding to 818409-818428 was synthesized, and ‘TATCGTAGTTATCTACACGAGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 60) which is an sgRNA that recognizes ‘TATCGTAGTTATCTACACGA’ (SEQ ID NO: 59) that is a portion corresponding to 818501-818520 was synthesized, and ‘CTACGTGAAAGGCGAGATCAGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 62) which is an sgRNA that recognizes ‘CTACGTGAAAGGCGAGATCA’ (SEQ ID NO: 61) that is a portion corresponding to 818606-818625 was synthesized to constitute the end portion.

### Preparation Example II-1-2

### Synthesis of 9 sgRNAs to Capture Cat Gene in EcNR2EcNR2 Genome

To capture the cat gene′ ATGGAGAAAAAAATCACTGGATATACCACCGTTGATATATCCCAATGGC ATCGTAAAGAACATTTTGAGGCATTTCAGTCAGTTGCTCAATGTACCTAT AACCAGACCGTTCAGCTGGATATTACGGCCTTTTTAAAGACCGTAAAGAA AAATAAGCACAAGTTTTATCCGGCCTTTATTCACATTCTTGCCCGCCTGAT GAATGCTCATCCGGAATTTCGTATGGCAATGAAAGACGGTGAGCTGGTG ATATGGGATAGTGTTCACCCTTGTTACACCGTTTTCCATGAGCAAACTGA AACGTTTTCATCGCTCTGGAGTGAATACCACGACGATTTCCGGCAGTTTC TACACATATATTCGCAAGATGTGGCGTGTTACGGTGAAAACCTGGCCTAT TTCCCTAAAGGGTTTATTGAGAATATGTTTTTCGTCTCAGCCAATCCCTGG GTGAGTTTCACCAGTTTTGATTTAAACGTGGCCAATATGGACAACTTCTT CGCCCCCGTTTTCACCATGGGCAAATATTATACGCAAGGCGACAAGGTGC TGATGCCGCTGGCGATTCAGGTTCATCATGCCGTCTGTGATGGCTTCCAT GTCGGCAGAATGCTTAATGAATTACAACAGTACTGCGATGAGTGGCAGG GCGGGGCGTAA′ 2864595-2865254 (SEQ ID NO: 63) in EcNR2EcNR2 genome, inventors extend 150 base pair at both ends of cat gene ‘CGCGGAATTCATGCTATCGACGTCGATATCTGGCGAAAATGAGACGTTG ATCGGCACGTAAGAGGTTCCAACTTTCACCATAATGAAATAAGATCACTA CCGGGCGTATTTTTTGAGTTATCGAGATTTTCAGGAGCTAAGGAAGCTAA AATGGAGAAAAAAATCACTGGATATACCACCGTTGATATATCCCAATGG CATCGTAAAGAACATTTTGAGGCATTTCAGTCAGTTGCTCAATGTACCTA TAACCAGACCGTTCAGCTGGATATTACGGCCTTTTTAAAGACCGTAAAGA AAAATAAGCACAAGTTTTATCCGGCCTTTATTCACATTCTTGCCCGCCTG ATGAATGCTCATCCGGAATTTCGTATGGCAATGAAAGACGGTGAGCTGGT GATATGGGATAGTGTTCACCCTTGTTACACCGTTTTCCATGAGCAAACTG AAACGTTTTCATCGCTCTGGAGTGAATACCACGACGATTTCCGGCAGTTT CTACACATATATTCGCAAGATGTGGCGTGTTACGGTGAAAACCTGGCCTA TTTCCCTAAAGGGTTTATTGAGAATATGTTTTTCGTCTCAGCCAATCCCTG GGTGAGTTTCACCAGTTTTGATTTAAACGTGGCCAATATGGACAACTTCT TCGCCCCCGTTTTCACCATGGGCAAATATTATACGCAAGGCGACAAGGTG CTGATGCCGCTGGCGATTCAGGTTCATCATGCCGTCTGTGATGGCTTCCA TGTCGGCAGAATGCTTAATGAATTACAACAGTACTGCGATGAGTGGCAG GGCGGGGCGTAATTTGATATCGAGCTCGTCAGCAGGCGCGCCTGTAATCA CACTGGCTCACCTTCGGGTGGGCCTTTCTGCGTTTAAAAAAAACGGGCCG GCGCGAACGCCGGCCCGCGGCCGCCACCCAGCTTTTGTTCCCTTTAGCGT CAGGCGCTGGAG’ 2864445-2865404 (SEQ ID NO: 64) in EcNR2EcNR2 genome for sufficiently capture both ends of gene and design 9 sgRNAs in the extended cat region for binding CRISPR-Cas complex. ‘GGCGAAAATGAGACGTTGATGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 66) which is an sgRNA that recognizes ‘GGCGAAAATGAGACGTTGAT’ (SEQ ID NO: 65) that is a portion corresponding to 2864476-2864495 was synthesized to constitute the front portion, and AGGAGCTAAGGAAGCTAAAAGTTTTAGAGCTAGAAATAGCAAGTTAAAA TAAGGCTAGTCCGTTATCAACTTGAAAAAGTGGCACCGAGTCGGTGCTTT TTTT′ (SEQ ID NO: 68) which is an sgRNA that recognizes ‘AGGAGCTAAGGAAGCTAAAA’ (SEQ ID NO: 67) that is a portion corresponding to 2864576-2864595 was synthesized, and ‘ATAACCAGACCGTTCAGCTGGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 70) which is an sgRNA that recognizes ‘ATAACCAGACCGTTCAGCTG’ (SEQ ID NO: 69) that is a portion corresponding to 2864692-2864711 was synthesized, and ‘GATGAATGCTCATCCGGAATGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 72) which is an sgRNA that recognizes ‘GATGAATGCTCATCCGGAAT’ (SEQ ID NO: 71) that is a portion corresponding to 2864792-2864811 was synthesized, and ‘TGAGCAAACTGAAACGTTTTGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 74) which is an sgRNA that recognizes ‘TGAGCAAACTGAAACGTTTT’ (SEQ ID NO: 73) that is a portion corresponding to 2864882-2864901 was synthesized, and ‘GGCCTATTTCCCTAAAGGGTGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 76) which is an sgRNA that recognizes ‘GGCCTATTTCCCTAAAGGGT’ (SEQ ID NO: 75) that is a portion corresponding to 2864987-2865006 was synthesized, and ‘ATATGGACAACTTCTTCGCCGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 78) which is an sgRNA that recognizes ‘ATATGGACAACTTCTTCGCC’ (SEQ ID NO: 77) that is a portion corresponding to 2865079-2865098 was synthesize, and ‘TCTGTGATGGCTTCCATGTCGTTTTAGAGCTAGAAATAGCAAGTTAAAAT AAGGCTAGTCCGTTATCAACTTGAAAAAGTGGCACCGAGTCGGTGCTTTT TTT’ (SEQ ID NO: 80) which is an sgRNA that recognizes ‘TCTGTGATGGCTTCCATGTC’ (SEQ ID NO: 79) that is a portion corresponding to 2865178-2865197 was synthesize, and ‘TTGATATCGAGCTCGTCAGCGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 82) which is an sgRNA that recognizes ‘TTGATATCGAGCTCGTCAGC’ (SEQ ID NO: 81) that is a portion corresponding to 2865256-2865275 was synthesized to constitute the end portion.

### Preparation Example II-2

### Preparation of dCas9 Protein to Capture Genetic Sequences Located at Multiple Sites

A dCas gene (mutanted Cas9 gene of Streptococcus pyogenes) was inserted into a pET28a vector which is a type of an E. coli expression vector. In this case, the portion of a vector sequence that is related to protein expression consists of a T7 promoter, a dCas9 gene, and a DNA sequence that expresses a histidine-tag (His-tag) for purification. This vector is a vector whose expression is controlled by a T7 RNA polymerase and a lac operator, occurs only in the presence of a T7 RNA polymerase, and increases significantly when the vector is incubated with isopropyl beta-D-1-thiogalactopyranoside (IPTG). The vector that was prepared as thus was introduced to E. coli (T7 Express Competent E. coli from NEB Inc.) having a T7 RNA polymerase to overexpress the dCas9 protein, and the protein was subsequently purified.

In purifying the dCas9 protein, first, the E. coli that overexpressed the protein was collected by centrifugation (3900 rpm, 10 mM) and the cell culture medium was completely discarded. Then, a lysis buffer (20 mM Tris-HCl at pH 8.0, 300 mM NaCl, 1 mg/mL lysozyme, lx phenylmethylsulfonyl fluoride (PMSF)) was added in a ratio of 1 mL lysis buffer/100 mL cell culture medium, and the E. coli was resuspended to be crushed by sonication (for total of 10 minutes; one cycle consists of crushing at 40% amplitude for 10 seconds and resting for 30 seconds). After sonication, the solution was centrifuged (13000 rpm, 10 mM) to obtain only a supernatant and was subsequently passed through a Ni-NTA resin to leave only a protein having His-tag on the resin. Then, the resin was washed with 5 mL of washing buffer (20 mm Tris-HCl at pH 8.0, 300 mM NaCl, 20 mm imidazole 1×PMSF) three times to remove unwanted proteins that are bound to the resin abnormally. Subsequently, only the wanted proteins were collected by passing an elution buffer (20 mm Tris-HCl at pH 8.0, 300 mM NaCl, 250 mM imidazole, 1×PMSF) 500 μL through the resin eight times to again obtain the proteins.

To use the purified proteins for capturing a genetic sequence, first, the solution should be replaced by a working buffer (50 mM Tris-HCl at pH 8.0, 200 mM KCl, 0.1 mM EDTA, 1 mM DTT, 0.5 mM PMSF, 20% glycerol) in which the proteins function. This is a process employing a dialysis method to simultaneously remove imidazole which is contained in the elution buffer in a significant amount and transfer the proteins to a solution that can keep the proteins in a more stable state. Among the eight solutions that were separately eluted, three solutions that contain eluted proteins totaling 1.5 mL were put in a dialysis cassette and then were subjected to a dialysis for 16 hours using 1 L of working buffer. The proteins that changed the composition of the solution were quantified by the Bradford assay.

### Preparation Example II-3

### Purification of Genome Sample for Capturing Target Nucleic Acid Sequences Located at Multiple Sites

For obtain a genome sample for capturing the target nucleic acid sequences located at multiple sites, Escherichia Coli EcNR2 strain were cultured and subsequently purified. Culture conditions included 30° C. and incubation Luria Broth(LB) as the culture medium. The cultured cells were harvested by centrifugation (3600 rpm, 10 min) for collected only the cells. Then, only genomes were purified using a Exgen Cell SV mini Kit from GeneAll Inc.

### Example II-1

### Simultaneous Capturing of Sheared Genetic Sequences Located at Multiple Sites by Attaching DNAs

480.7 ng of the sgRNA library prepared by the preparation example II-1 was used with 2248.3 ng of the dCas9 protein prepared by the preparation example II-2 under aforementioned conditions of a Cas9 working buffer. After the volume was set to 20 μL, they were allowed to react for 1 hour at 37° C. to simultaneously capture genetic sequences located at multiple sites. To confirm if the simultaneous capturing of genetic sequences located at multiple sites had been successful, sequencing of the captured sequence was performed. Specifically, target nucleic acids containing EcNR2 genome was sheared before CRISPR-Cas attaching capture. Adaptor sequences for next-generation sequencing equipment were attached to sheared EcNR2 genome by SPARK DNA sample prep Kit(Enzymatics. Inc). Using a USER enzyme, the DNA fragments to which adapters are attached cut uracil that existed in an adapter DNA and amplified the captured sequences using a universal sequence primer and an index sequence available from Illumina Inc. The amplified sequences were separated by size using an agarose gel and, in this case, only those of desired sizes were selected for purification using a MinElute PCR Purification kit from QIAGEN Inc.

After next-generation adaptor attached sheared EcNR2 genome was prepared, mixing dCas9 and sgRNA library for construct CRISPR complexes, and add pre-treated EcNR2 genome for attaching complexes to target sequence in fragments.

After the attaching reaction, for sorting target nucleic acid sequences, inventors use Ni-NTA magnet bead for binding histidine tag at dCas9 in CRISPR complexes and purify the CRISPR-Cas-target nucleic acid complexes. Ni-NTA purified target nucleic acids were amplified using a universal sequence primer and an index sequence available from Illumina Inc. The amplified sequences were separated by size using an agarose gel and, in this case, only those of desired sizes were selected for purification using a MinElute PCR Purification kit from QIAGEN Inc. Subsequently, the sequencing information was obtained using a next-generation NextSeq sequencing system. The obtained sequencing information was analyzed by programs such as a self-produced Python program, BWA, or the like to confirm if desired sequences had been captured, and it was confirmed that desired genetic sequences had been simultaneously captured.

To exemplify some of the above, the sequencing result of ‘CACGAGTGGGTTACATCGAACTGGATCTCAACAGCGGTAAGATCCTTGA GAGTTTTCGCCCCGAAGAACGTTTTCCAATGATGAGCACTTTTAAAGTTC TGCTATGTGGCGCGGTATTATCCCGTATTGACGCCGGGCA’(SEQ ID NO: 83) confirmed that the genetic sequence of ‘CACGAGTGGGTTACATCGAACTGGATCTCAACAGCGGTAAGATCCTTGA GAGTTTTCGCCCCGAAGAACGTTTTCCAATGATGAGCACTTTTAAAGTTC TGCTATGTGGCGCGGTATTATCCCGTATTGACGCCGGGCA’ (SEQ ID NO: 84) corresponding to part of bla gene region (SEQ ID NO: 39) of EcNR2 817855-817993 had been captured by sgRNA ‘CGAAGAACGTTTTCCAATGAGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 48), which was preparation example II-1-1.

In addition, the sequencing result of ‘CTGCGCTCGGCCCTTCCGGCTGGCTGGTTTATTGCTGATAAATCTGGAGC CGGTGAGCGTGGGTCTCGCGGTATCATTGCAGCACTGGGGCCAGATGGT AAGCCCTCCCGTATCGTAGTTATCTACACGAC’ (SEQ ID NO: 85) confirmed that the genetic sequence of the genetic sequence of ‘CTGCGCTCGGCCCTTCCGGCTGGCTGGTTTATTGCTGATAAATCTGGAGC CGGTGAGCGTGGGTCTCGCGGTATCATTGCAGCACTGGGGCCAGATGGT AAGCCCTCCCGTATCGTAGTTATCTACACGAC’ of SEQ ID NO: 86 corresponding to part of bla gene region (SEQ ID NO: 39) of EcNR2 818391-818521 had been accurately captured by sgRNA′ GCTGGCTGGTTTATTGCTGAGTTTTAGAGCTAGAAATAGCAAGTTAAAAT AAGGCTAGTCCGTTATCAACTTGAAAAAGTGGCACCGAGTCGGTGCTTTT TTT′, SEQ ID NO: 58 or sgRNA′ TATCGTAGTTATCTACACGAGTTTTAGAGCTAGAAATAGCAAGTTAAAAT AAGGCTAGTCCGTTATCAACTTGAAAAAGTGGCACCGAGTCGGTGCTTTT TTT′ which was SEQ ID NO: 60 of the preparation example II-1-1.

In the case of another region to be captured, the sequencing result of ‘CGTAAAGAACATTTTGAGGCATTTCAGTCAGTTGCTCAATGTACCTAT AACCAGACCGTTCAGCTGGATATTACGGCCTTTTTAAAGACCGTAAAG AAAAATAAGCACAAGTTTTATCCGGCC’(SEQ ID NO: 87) confirmed that the genetic sequence of ‘CGTAAAGAACATTTTGAGGCATTTCAGTCAGTTGCTCAATGTACCTAT AACCAGACCGTTCAGCTGGATATTACGGCCTTTTTAAAGACCGTAAAG AAAAATAAGCACAAGTTTTATCCGGCC’ (SEQ ID NO: 88) which is the portion corresponding to cat gene region (SEQ ID NO: 63) of EcNR2 2864646-2864768, had been captured by sgRNA ATAACCAGACCGTTCAGCTGGTTTTAGAGCTAGAAATAGCAAGTTAAA ATAAGGCTAGTCCGTTATCAACTTGAAAAAGTGGCACCGAGTCGGTGC TTTTTTT′ (SEQ ID NO: 70) which was preparation example II-1-2.

Also the sequencing result of ‘GCTCTGGAGTGAATACCACGACGATTTCCGGCAGTTTCTACACATATATT CGCAAGATGTGGCGTGTTACGGTGAAAACCTGGCCTATTTCCCTAAAGGG TTTATTGAGAATATGTTTTTCGTCTCAGCCAATCCCTGGGTGAGTTTCACC’ (SEQ ID NO: 89) confirmed that the genetic sequence of ‘GCTCTGGAGTGAATACCACGACGATTTCCGGCAGTTTCTACACATATATT CGCAAGATGTGGCGTGTTACGGTGAAAACCTGGCCTATTTCCCTAAAGGG TTTATTGAGAATATGTTTTTCGTCTCAGCCAATCCCTGGGTGAGTTTCACC’ (SEQ ID NO: 90) corresponding to part of cat gene region (SEQ ID NO: 63) of EcNR2 ‘2864906-2865056 had been accurately be captured by sgRNA ‘GGCCTATTTCCCTAAAGGGTGTTTTAGAGCTAGAAATAGCAAGTTAAAA TAAGGCTAGTCCGTTATCAACTTGAAAAAGTGGCACCGAGTCGGTGCTTT TTTT’ (SEQ ID NO: 76), which was preparation example II-1-2 was identified.

As shown in the results, the simultaneous capturing of a variety of genetic sequences was successfully achieved.

1. A method of capturing a target nucleic acid sequence in genome sequencing, the method comprising:
treating a genome sample including a target nucleic acid sequence, with a plurality of CRISPR systems that can cut at both ends of the target nucleic acid sequence or can complementarily bind to CRISPR complex-binding sequence within the target nucleic acid sequence, and
sorting the target nucleic acid sequences from fragments of genome sample or PCR amplification products thereof,
wherein one or more target nucleic acid sequences within genome are captured simultaneously.

2. A method of capturing a target nucleic acid sequence in genome sequencing, the method comprising:
treating a genome sample including a target nucleic acid sequence, with a plurality of CRISPR systems that can cut at both ends of the target nucleic acid sequence,
sorting the target nucleic acid sequence from fragments of genome sample or PCR amplification products thereof,
wherein one or more target nucleic acid sequences within genome are captured simultaneously.

3. The method of claim 2, the method comprising:
treating a genome sample including a target nucleic acid sequence, with a plurality of CRISPR systems that can cut at both ends of the target nucleic acid sequence and additionally one or more CRISPR systems that can cut at one or more predetermined sites within the target nucleic acid sequences,
sorting the target nucleic acid sequence from fragments of genome sample or PCR amplification products thereof,
wherein one or more target nucleic acid sequences within genome are captured simultaneously.

4. A method of capturing a target nucleic acid sequence in genome sequencing, the method comprising:
treating a genome sample including a target nucleic acid sequence, with a plurality of CRISPR systems that can complementarily bind to CRISPR complex-binding sequence within the target nucleic acid sequence, and
sorting the target nucleic acid sequence from fragments of genome sample or PCR amplification products thereof,
wherein one or more target nucleic acid sequences within genome are captured simultaneously.

5. The method of claim 1, wherein the CRISPR system includes an sgRNA and a CRISPR enzyme; or a crRNA, a tracrRNA and a CRISPR enzyme.

6. The method of claim 1, wherein the CRISPR system is an sgRNA and a CRISPR enzyme.

7. The method of claim 6, wherein the sgRNA is an sgRNA library obtained from a template DNA by in vitro transcription.

8. The method of claim 7, wherein the template DNA comprises:
a promoter that can bind with an RNA polymerase to initiate transcription; and
a DNA sequence that codes the sgRNA.

9. The method of claim 5, wherein the CRISPR enzyme is a type II CRISPR system enzyme.

10. The method of claim 5, wherein the CRISPR enzyme is a Cas9 enzyme.

11. The method of claim 10, wherein the Cas9 enzyme is an ortholog of Cas9, which originates from a genus of a microorganism selected from the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter.

12. The method of claim 1, wherein the target nucleic acid sequence is DNA, RNA or PNA.

13. The method of claim 1, wherein the target nucleic acid sequence originates from an animal or a plant.

14. The method of claim 2, wherein the CRISPR enzyme is a wild type of CRISPR enzyme.

15. The method of claim 5, wherein the CRISPR enzyme is a mutated CRISPR enzyme.

16. The method of claim 1, wherein the selection of the target nucleic acid sequence is performed by isolating based on size of nucleic acid sequence or by using probe.


SEQUENCE LISTING (90 sequences)

SEQ ID NO: 1 - DNA, 25 bp, Artificial Sequence
  T7 promoter
ggattctaatacgactcactatagg

SEQ ID NO: 2 - DNA, 18 bp, Artificial Sequence
  CRISPR complex-binding sequence1, N is one selected from A, T, C, and G.
nnnnnnnnnnnnnnnnnn

SEQ ID NO: 3 - DNA, 83 bp, Artificial Sequence
  sgRNA scaffold
gttttagagctagaaatagcaagttaaaataaggctagtccgttatcaacttgaaaaagtggcaccgagtcggtgcttttttt

SEQ ID NO: 4 - DNA, 126 bp, Artificial Sequence
  template DNA sequence, N is one selected from A, T, C, and G.
ggattctaatacgactcactataggnnnnnnnnnnnnnnnnnngttttagagctagaaatagcaagttaaaataaggctagtccgttatcaacttgaaaaagtggcaccgagtcggtgcttttttt

SEQ ID NO: 5 - DNA, 243 bp, Homo sapiens
ggatcggactctttccgtcacccgtttgcacctctgcagctgtcaggagcgggtcaggtgcggaaagcggtgcggaggtggcgctcataggttacaggggtcagggtctggggctggccgtggtcttcagttaccgccgagcgtgcgggatccttctgcgcttgccgcctccacgtggcacaggccaaggcgtggccagatgggtagatgggtttgttgggtggttgctagcagtttccacgt

SEQ ID NO: 6 - DNA, 18 bp, Homo sapiens
ggaggatcggactctttc

SEQ ID NO: 7 - DNA, 101 bp, Artificial Sequence
  sgRNA for recognizing the CRISPR complex- binding sequence of position 1448011-1448028 of chromosome 1
gaaagagtccgatcctccgttttagagctagaaatagcaagttaaaataaggctagtccgttatcaacttgaaaaagtggcaccgagtcggtgcttttttt

SEQ ID NO: 8 - DNA, 18 bp, Homo sapiens
cgtaacaagggaagcgta

SEQ ID NO: 9 - DNA, 101 bp, Artificial Sequence
  sgRNA for recognizing the CRISPR complex- binding sequence of position 1448254-1448271 of chromosome 1
tacgcttcccttgttacggttttagagctagaaatagcaagttaaaataaggctagtccgttatcaacttgaaaaagtggcaccgagtcggtgcttttttt

SEQ ID NO: 10 - DNA, 267 bp, Homo sapiens
cagaggttgcagtttctgagaaacacactgaaaatcctccataagtgatttagaccacgcaaaaacaagagacaactctcacctgagctgaaatggttcgctgaaaggtttttccagttgatgtttcattagagacattactctgtggtgtccagtaatgttctgacatctgagatgaaaggtcaaaaatgccatcagaggtgacaaataagcccccatgggttcacagtttctaccattagatattgagtcttaaaagcatcccaa

SEQ ID NO: 11 - DNA, 18 bp, Homo sapiens
tcatacctctcttctcag

SEQ ID NO: 12 - DNA, 101 bp, Artificial Sequence
  sgRNA for recognizing the CRISPR complex- binding sequence of position 55537893-55537910 of chromosome 1
tcatacctctcttctcaggttttagagctagaaatagcaagttaaaataaggctagtccgttatcaacttgaaaaagtggcaccgagtcggtgcttttttt

SEQ ID NO: 13 - DNA, 18 bp, Homo sapiens
ttaaaagcatcccaagta

SEQ ID NO: 14 - DNA, 101 bp, Artificial Sequence
  sgRNA for recognizing the CRISPR complex- binding sequence of position 55538160-55538177 of chromosome 1
ttaaaagcatcccaagtagttttagagctagaaatagcaagttaaaataaggctagtccgttatcaacttgaaaaagtggcaccgagtcggtgcttttttt

SEQ ID NO: 15 - DNA, 504 bp, Homo sapiens
acagggggaaaaccctatgaatgtcatgaatgtgggaagaccttctataagaattcagacctcattaaacatcaaagaattcatacaggggagagaccttatggatgtcatgaatgtgggaaatccttcagtgaaaagtcaacccttactcaacatcaaagaacgcacacaggggagaaaccatatgaatgtcatgaatgtgggaaaaccttctcatttaagtcagtccttactgtgcatcagaaaacacacacaggggagaagccctatgaatgctatgcatgtgggaaagcctttctcagaaaatcagacctcattaaacatcaaagaatacacacaggtgaaaaaccttatgaatgtaatgaatgtgggaagtcattctctgagaagtcaacccttactaaacatctaagaactcacacaggtgagaaaccttatgaatgtattcagtgtggaaaatttttctgctactactccggtttcacagaacatctgagaagacac

SEQ ID NO: 16 - DNA, 18 bp, Homo sapiens
tcagagaacacacacagg

SEQ ID NO: 17 - DNA, 101 bp, Artificial sequence
  sgRNA for recognizing the CRISPR complex- binding sequence of position 38406946-38406963 of chromosome 10
tcagagaacacacacagggttttagagctagaaatagcaagttaaaataaggctagtccgttatcaacttgaaaaagtggcaccgagtcggtgcttttttt

SEQ ID NO: 18 - DNA, 18 bp, Homo sapiens
gcatcagaaaacacacac

SEQ ID NO: 19 - DNA, 101 bp, Artificial sequence
  sgRNA for recognizing the CRISPR complex- binding sequence of position 38407195-38407212 of chromosome 10
gcatcagaaaacacacacgttttagagctagaaatagcaagttaaaataaggctagtccgttatcaacttgaaaaagtggcaccgagtcggtgcttttttt

SEQ ID NO: 20 - DNA, 18 bp, Homo sapiens
acatctgagaagacacac

SEQ ID NO: 21 - DNA, 101 bp, Artificial Sequence
  sgRNA for recognizing the CRISPR complex- binding sequence of position 38407447-38407464 of chromosome 10
acatctgagaagacacacgttttagagctagaaatagcaagttaaaataaggctagtccgttatcaacttgaaaaagtggcaccgagtcggtgcttttttt

SEQ ID NO: 22 - DNA, 260 bp, Homo sapiens
ttaagggttaagtaattacacatctgttttgctttttcttccttctatagtcttaacatagtactctacccacaggtggtgacaggaaggaaattggatgtggaatgtggaaaggtggaaacctctaccttgaacaggttgatgttgtcgatctggctctggaagagaaagtcgttgatagtcttcagctccatccctgagaacaaacacatgaagggccttgggagcttcaccctaagcctcaggtttcagtcccaggg

SEQ ID NO: 23 - DNA, 18 bp, Homo sapiens
acaggcgtgttgcgttaa

SEQ ID NO: 24 - DNA, 101 bp, Artificial Sequence
  sgRNA for recognizing the CRISPR complex- binding sequence of position 9580087-9580104  of chromosome 12
acaggcgtgttgcgttaagttttagagctagaaatagcaagttaaaataaggctagtccgttatcaacttgaaaaagtggcaccgagtcggtgcttttttt

SEQ ID NO: 25 - DNA, 18 bp, Homo sapiens
agggttaagctcggaagt

SEQ ID NO: 26 - DNA, 101 bp, Artificial Sequence
  sgRNA for recognizing the CRISPR complex- binding sequence of position 9580357-9580374 of chromosome 12
acttccgagcttaaccctgttttagagctagaaatagcaagttaaaataaggctagtccgttatcaacttgaaaaagtggcaccgagtcggtgcttttttt

SEQ ID NO: 27 - DNA, 151 bp, Artificial Sequence
  sequencing result1 for SEQ ID NO.5
ggatcggactctttccgtcacccgtttgcacctctgcagctgtcaggagcgggtcaggtgcggaaagcggtgcggaggtggcgctcataggttacaggggtcagggtctggggctggccgtggtcttcagttaccgccgagcgtgcgggat

SEQ ID NO: 28 - DNA, 151 bp, Artificial Sequence
  sequencing result2 for SEQ ID NO.5
tacaggggtcagggtctggggctggccgtggtcttcagttaccgccgagcgtgcgggatccttctgcgcttgccgcctccacgtggcacaggccaaggcgtggccagatgggtagatgggtttgttgggtggttgctagcagtttccacgt

SEQ ID NO: 29 - DNA, 151 bp, Artificial Sequence
  sequencing result1 for SEQ ID NO.10
cagaggttgcagtttctgagaaacacactgaaaatcctccataagtgatttagaccacgcaaaaacaagagacaactctcacctgagctgaaatggttcgctgaaaggtttttccagttgatgtttcattagagacattactctgtggtgt

SEQ ID NO: 30 - DNA, 151 bp, Artificial Sequence
  sequencing result2 for SEQ ID NO.10
gttgatgtttcattagagacattactctgtggtgtccagtaatgttctgacatctgagatgaaaggtcaaaaatgccatcagaggtgacaaataagcccccatgggttcacagtttctaccattagatattgagtcttaaaagcatcccaa

SEQ ID NO: 31 - DNA, 151 bp, Artificial Sequence
  sequencing result1 for SEQ ID NO.15
agggggaaaaccctatgaatgtcatgaatgtgggaagaccttctataagaattcagacctcattaaacatcaaagaattcatacaggggagagaccttatggatgtcatgaatgtgggaaatccttcagtgaaaagtcaacccttactcaa

SEQ ID NO: 32 - DNA, 151 bp, Artificial Sequence
  sequencing result2 for SEQ ID NO.15
tggatgtcatgaatgtgggaaatccttcagtgaaaagtcaacccttactcaacatcaaagaacgcacacaggggagaaaccatatgaatgtcatgaatgtgggaaaaccttctcatttaagtcagtccttactgtgcatcagaaaacacac

SEQ ID NO: 33 - DNA, 151 bp, Artificial Sequence
  sequencing result3 for SEQ ID NO.15
acaggggagaagccctatgaatgctatgcatgtgggaaagcctttctcagaaaatcagacctcattaaacatcaaagaatacacacaggtgaaaaaccttatgaatgtaatgaatgtgggaagtcattctctgagaagtcaacccttacta

SEQ ID NO: 34 - DNA, 151 bp, Artificial Sequence
  sequencing result4 for SEQ ID NO.15
atgaatgtaatgaatgtgggaagtcattctctgagaagtcaacccttactaaacatctaagaactcacacaggtgagaaaccttatgaatgtattcagtgtggaaaatttttctgctactactccggtttcacagaacatctgagaagaca

SEQ ID NO: 35 - DNA, 151 bp, Artificial Sequence
  sequencing result1 for SEQ ID NO.22
taagggttaagtaattacacatctgttttgctttttcttccttctatagtcttaacatagtactctacccacaggtggtgacaggaaggaaattggatgtgcaatgtggaaaggtggaaacctctaccttgaacaggttgatgttgtcgat

SEQ ID NO: 36 - DNA, 151 bp, Artificial Sequence
  sequencing result2 for SEQ ID NO.22
ggaaaggtggaaacctctaccttgaacaggttgatgttgtcgatctggctctggaagagaaagtcgttgatagtcttcagctccatccctgagaacaaacacatgaagggccttgggagcttcaccctaagcctcaggtttcagtcccagg

SEQ ID NO: 37 - DNA, 20 bp, Artificial Sequence
  CRISPR complex-binding sequence2, N is one selected from A, T, C, and G.
nnnnnnnnnnnnnnnnnnnn

SEQ ID NO: 38 - DNA, 128 bp, Artificial sequence
  Template DNA sequence 2
ggattctaatacgactcactataggnnnnnnnnnnnnnnnnnnnngttttagagctagaaatagcaagttaaaataaggctagtccgttatcaacttgaaaaagtggcaccgagtcggtgcttttttt

SEQ ID NO: 39 - DNA, 861 bp, Escherichia coli
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 40 - DNA, 1161 bp, Escherichia coli
ttattcggccttgaattgatcatatgcggattagaaaaacaacttaaatgtgaaagtgggtcttaacagttcctggatatccggatgaaggcacgaacccagtggacataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaatttgtccactacgtgaaaggcgagatcaccaaggtagtcggcaaataatgtctaacaattcgttcaagccgacggatatcgagctcgcttggactcctgttgatagatccagtaatgacctcagaactccatctggatttgttcagaacg

SEQ ID NO: 41 - DNA, 20 bp, Escherichia coli
aaacaacttaaatgtgaaag

SEQ ID NO: 42 - DNA, 103 bp, Artificial sequence
  sgRNA recognizing position 817623-817642 of bla gene
aaacaacttaaatgtgaaaggttttagagctagaaatagcaagttaaaataaggctagtccgttatcaacttgaaaaagtggcaccgagtcggtgcttttttt

SEQ ID NO: 43 - DNA, 20 bp, Escherichia coli
tgcttcaataatattgaaaa

SEQ ID NO: 44 - DNA, 103 bp, Artificial sequence
  sgRNA recognizing position 817708-817727 of bla gene
tgcttcaataatattgaaaagttttagagctagaaatagcaagttaaaataaggctagtccgttatcaacttgaaaaagtggcaccgagtcggtgcttttttt

SEQ ID NO: 45 - DNA, 20 bp, Escherichia coli
ttttgctcacccagaaacgc

SEQ ID NO: 46 - DNA, 103 bp, Artificial sequence
  sgRNA recognizing position 817799-817818 of bla gene
ttttgctcacccagaaacgcgttttagagctagaaatagcaagttaaaataaggctagtccgttatcaacttgaaaaagtggcaccgagtcggtgcttttttt

SEQ ID NO: 47 - DNA, 20 bp, Escherichia coli
cgaagaacgttttccaatga

SEQ ID NO: 48 - DNA, 103 bp, Artificial sequence
  sgRNA recognizing position 817916-817935 of bla gene
cgaagaacgttttccaatgagttttagagctagaaatagcaagttaaaataaggctagtccgttatcaacttgaaaaagtggcaccgagtcggtgcttttttt

SEQ ID NO: 49 - DNA, 20 bp, Escherichia coli
catacactattctcagaatg

SEQ ID NO: 50 - DNA, 103 bp, Artificial sequence
  sgRNA recognizing position 818012-818031 of bla gene
catacactattctcagaatggttttagagctagaaatagcaagttaaaataaggctagtccgttatcaacttgaaaaagtggcaccgagtcggtgcttttttt

SEQ ID NO: 51 - DNA, 20 bp, Escherichia coli
taaccatgagtgataacact

SEQ ID NO: 52 - DNA, 103 bp, Artificial sequence
  sgRNA recognizing position 818110-818129 of bla gene
taaccatgagtgataacactgttttagagctagaaatagcaagttaaaataaggctagtccgttatcaacttgaaaaagtggcaccgagtcggtgcttttttt

SEQ ID NO: 53 - DNA, 20 bp, Escherichia coli
tgatcgttgggaaccggagc

SEQ ID NO: 54 - DNA, 103 bp, Artificial sequence
  sgRNA recognizing position 818216-818235 of bla gene
tgatcgttgggaaccggagcgttttagagctagaaatagcaagttaaaataaggctagtccgttatcaacttgaaaaagtggcaccgagtcggtgcttttttt

SEQ ID NO: 55 - DNA, 20 bp, Escherichia coli
acgttgcgcaaactattaac

SEQ ID NO: 56 - DNA, 103 bp, Artificial sequence
  sgRNA recognizing position 818295-818314 of bla gene
acgttgcgcaaactattaacgttttagagctagaaatagcaagttaaaataaggctagtccgttatcaacttgaaaaagtggcaccgagtcggtgcttttttt

SEQ ID NO: 57 - DNA, 20 bp, Escherichia coli
gctggctggtttattgctga

SEQ ID NO: 58 - DNA, 103 bp, Artificial sequence
  sgRNA recognizing position 818409-818428 of bla gene
gctggctggtttattgctgagttttagagctagaaatagcaagttaaaataaggctagtccgttatcaacttgaaaaagtggcaccgagtcggtgcttttttt

SEQ ID NO: 59 - DNA, 20 bp, Escherichia coli
tatcgtagttatctacacga

SEQ ID NO: 60 - DNA, 103 bp, Artificial sequence
  sgRNA recognizing position 818501-818520 of bla gene
tatcgtagttatctacacgagttttagagctagaaatagcaagttaaaataaggctagtccgttatcaacttgaaaaagtggcaccgagtcggtgcttttttt

SEQ ID NO: 61 - DNA, 20 bp, Escherichia coli
ctacgtgaaaggcgagatca

SEQ ID NO: 62 - DNA, 103 bp, Artificial sequence
  sgRNA recognizing position 818606-818625 of bla gen
ctacgtgaaaggcgagatcagttttagagctagaaatagcaagttaaaataaggctagtccgttatcaacttgaaaaagtggcaccgagtcggtgcttttttt

SEQ ID NO: 63 - DNA, 660 bp, Escherichia coli
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaa

SEQ ID NO: 64 - DNA, 960 bp, Escherichia coli
cgcggaattcatgctatcgacgtcgatatctggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttgatatcgagctcgtcagcaggcgcgcctgtaatcacactggctcaccttcgggtgggcctttctgcgtttaaaaaaaacgggccggcgcgaacgccggcccgcggccgccacccagcttttgttccctttagcgtcaggcgctggag

SEQ ID NO: 65 - DNA, 20 bp, Escherichia coli
ggcgaaaatgagacgttgat

SEQ ID NO: 66 - DNA, 103 bp, Artificial sequence
  sgRNA recognizing position 2864476-2864495 of cat gene
ggcgaaaatgagacgttgatgttttagagctagaaatagcaagttaaaataaggctagtccgttatcaacttgaaaaagtggcaccgagtcggtgcttttttt

SEQ ID NO: 67 - DNA, 20 bp, Escherichia coli
aggagctaaggaagctaaaa

SEQ ID NO: 68 - DNA, 103 bp, Artificial sequence
  sgRNA recognizing position 2864576-2864595 of cat gene
aggagctaaggaagctaaaagttttagagctagaaatagcaagttaaaataaggctagtccgttatcaacttgaaaaagtggcaccgagtcggtgcttttttt

SEQ ID NO: 69 - DNA, 20 bp, Escherichia coli
ataaccagaccgttcagctg

SEQ ID NO: 70 - DNA, 103 bp, Artificial sequence
  sgRNA recognizing position 2864692-2864711 of cat gene
ataaccagaccgttcagctggttttagagctagaaatagcaagttaaaataaggctagtccgttatcaacttgaaaaagtggcaccgagtcggtgcttttttt

SEQ ID NO: 71 - DNA, 20 bp, Escherichia coli
gatgaatgctcatccggaat

SEQ ID NO: 72 - DNA, 103 bp, Artificial sequence
  sgRNA recognizing position 2864792-2864811 of cat gene
gatgaatgctcatccggaatgttttagagctagaaatagcaagttaaaataaggctagtccgttatcaacttgaaaaagtggcaccgagtcggtgcttttttt

SEQ ID NO: 73 - DNA, 20 bp, Escherichia coli
tgagcaaactgaaacgtttt

SEQ ID NO: 74 - DNA, 103 bp, Artificial sequence
  sgRNA recognizing position 2864882-2864901 of cat gene
tgagcaaactgaaacgttttgttttagagctagaaatagcaagttaaaataaggctagtccgttatcaacttgaaaaagtggcaccgagtcggtgcttttttt

SEQ ID NO: 75 - DNA, 20 bp, Escherichia coli
ggcctatttccctaaagggt

SEQ ID NO: 76 - DNA, 103 bp, Artificial sequence
  sgRNA recognizing position 2864987-2865006 of cat gene
ggcctatttccctaaagggtgttttagagctagaaatagcaagttaaaataaggctagtccgttatcaacttgaaaaagtggcaccgagtcggtgcttttttt

SEQ ID NO: 77 - DNA, 20 bp, Escherichia coli
atatggacaacttcttcgcc

SEQ ID NO: 78 - DNA, 103 bp, Artificial sequence
  sgRNA recognizing position 2865079-2865098 of cat gene
atatggacaacttcttcgccgttttagagctagaaatagcaagttaaaataaggctagtccgttatcaacttgaaaaagtggcaccgagtcggtgcttttttt

SEQ ID NO: 79 - DNA, 20 bp, Escherichia coli
tctgtgatggcttccatgtc

SEQ ID NO: 80 - DNA, 103 bp, Artificial sequence
  sgRNA recognizing position 2865178-2865197 of cat gene
tctgtgatggcttccatgtcgttttagagctagaaatagcaagttaaaataaggctagtccgttatcaacttgaaaaagtggcaccgagtcggtgcttttttt

SEQ ID NO: 81 - DNA, 20 bp, Escherichia coli
ttgatatcgagctcgtcagc

SEQ ID NO: 82 - DNA, 103 bp, Artificial sequence
  sgRNA sequence position 2865256-2865275 of cat gene
ttgatatcgagctcgtcagcgttttagagctagaaatagcaagttaaaataaggctagtccgttatcaacttgaaaaagtggcaccgagtcggtgcttttttt

SEQ ID NO: 83 - DNA, 139 bp, Artificial sequence
  sequencing result1 of bla gene
cacgagtgggttacatcgaactggatctcaacagcggtaagatccttgagagttttcgccccgaagaacgttttccaatgatgagcacttttaaagttctgctatgtggcgcggtattatcccgtattgacgccgggca

SEQ ID NO: 84 - DNA, 139 bp, Escherichia coli; identical to SEQ ID NO: 83

SEQ ID NO: 85 - DNA, 131 bp, Artificial sequence
  sequencing result2 of bla gene
ctgcgctcggcccttccggctggctggtttattgctgataaatctggagccggtgagcgtgggtctcgcggtatcattgcagcactggggccagatggtaagccctcccgtatcgtagttatctacacgac

SEQ ID NO: 86 - DNA, 131 bp, Escherichia coli; identical to SEQ ID NO: 85

SEQ ID NO: 87 - DNA, 123 bp, Artificial sequence
  sequencing result1 of cat gene
cgtaaagaacattttgaggcatttcagtcagttgctcaatgtacctataaccagaccgttcagctggatattacggcctttttaaagaccgtaaagaaaaataagcacaagttttatccggcc

SEQ ID NO: 88 - DNA, 123 bp, Escherichia coli; identical to SEQ ID NO: 87

SEQ ID NO: 89 - DNA, 151 bp, Artificial sequence
  sequencing result2 of cat gene
gctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcacc

SEQ ID NO: 90 - DNA, 151 bp, Escherichia coli; identical to SEQ ID NO: 89